(19) United States
(12) Patent Application Publication
(10) Pub. No.: US 2008/0177288 A1
(43) Pub. Date: Jul. 24, 2008

(54) Remote Suturing Device

(76) Inventor: Mark A. Carlson, Omaha, NE (US)

Correspondence Address: DILLON & YUDELL LLP, 8911 NORTH CAPITAL OF TEXAS HWY, SUITE 2110, AUSTIN, TX, 78759, US

(21) Appl. No.: 11/624,786

(22) Filed: Jan. 19, 2007

(51) Int. Cl. A61B 17/04

(52) U.S. Cl. 606/144

(57) ABSTRACT

A surgical incision suturing device particularly suited for minimally invasive surgical procedures integrates an elongated member such as a trocar with a suture deployment device. In one embodiment, the suturing device of the present invention includes an elongated member having a distal end adapted for placement within a surgical site. A pair of needle assemblies is disposed in substantially diametric opposition on the outside surface of the elongated member. Each of the needle assemblies includes a suture anchor coupled to the distal end of a needle shaft. In a further aspect, each of the needle assemblies is detachably coupled to the elongated member and biased to pivot radially outward from the elongated member at the detachable coupling.

### BACKGROUND OF THE INVENTION

1. Technical Field

The present invention relates generally to a device for suturing punctures or incisions extending into the peritoneum of a body and, particularly, to a suturing device for positioning and deploying suture anchors to seal such a puncture or incision.

2. Description of the Related Art

Many devices and techniques for suturing surgical wounds are known in the art. A primary aim in designing such devices/techniques is to assist surgeons in the suturing process, which is both time consuming and difficult due to the precision handwork and dexterity required to place sutures often in relatively inaccessible surgical sites. The problem of limited accessibility particularly arises when suturing a minimally invasive surgical site such as one resulting from laparoscopic or endoscopic surgery. Minimally invasive surgery is performed utilizing relatively small surgical instruments that are inserted into an internal body cavity through a port instrument, such as a trocar. As is known in the art, a trocar is generally an instrument comprising a hollow tubular sheath having a sharp cutting edge disposed on its distal end for piercing and passing through the external tissue layers over the surgical site. Following incision and placement of the trocar, surgical instruments such as laparoscopic graspers are passed through the trocar to the surgical site for subsequent surgical manipulation.

Prior to breaching the target internal surgical site, the trocar puncture/incision passes through the musculoaponeurotic layer or “fascia” comprising connective tissue disposed below the skin and subcutaneous fat layer. The fascia provides the primary structural strength of the abdominal wall and is vulnerable to trocar-site (referred to herein alternatively as “port-site”) herniation if not properly closed following surgery.

The increasing complexity of minimally invasive surgical procedures has resulted in a need for larger diameter trocars, some having outside diameters of up to 12 mm or larger. Unless closed properly, larger diameter trocar punctures/incisions may not heal satisfactorily, possibly allowing herniation through the resultant fascial defect. Other factors affecting the probability of trocar-site herniation include extent of port-site manipulation during the procedure and patient obesity.

Given the potential for hernias, the trocar-site incision must be securely closed in a manner adequately binding the breached fascial layer. Bioabsorbable sutures are the most common binding agent, with the sutures passing through opposing fascial tissue edges and tied to hold the more deeply buried portions of the edge of the wound together. However, the relative inaccessibility of a minimally invasive surgical site creates particular problems for accurate and reliable suturing since the fascial layer is more difficult to access than the target tissue layers in a typical “open” surgical site. Due to the limited view of the fascial layer and the risk of damage to abdominal organs, manual suturing techniques typically place sutures only through the outer layers of fascia.

To address problems with conventional unguided hand suturing, another manual incision closure technique used for trocar site incisions requires surgeons to laparoscopically grasp and manipulate sutures. While overcoming some of the aforementioned problems associated with unguided hand suturing, laparoscopically guided suturing is very tedious and time-consuming.

A variety of other trocar incision suturing techniques and devices are known in which the suturing needle is mechanically driven by a specialized suturing device. Such devices typically include curved or otherwise upwardly pointing needles pivotally positioned on a suture deployment shaft. These devices require complex, dynamically cooperative features for directing the suture needles and are relatively cumbersome in application.

It can therefore be appreciated that a need exists for an improved suturing device that addresses the foregoing problems with conventional devices/techniques for closing wounds incident to minimally invasive surgical procedures. The present invention addresses these as well as other needs unaddressed by prior art.

### SUMMARY OF THE INVENTION

A surgical incision suturing device that integrates an elongated member such as a trocar with a suture deployment device and is particularly suited for minimally invasive surgical procedures is disclosed herein. In one embodiment, the suturing device of the present invention includes an elongated member having a distal end adapted for placement within a surgical site. A pair of needle assemblies is disposed in substantially diametric opposition on the outside surface of the elongated member. Each of the needle assemblies includes a suture anchor coupled to the distal end of a needle shaft. In a further aspect, each of the needle assemblies is detachably coupled to the elongated member and biased to pivot radially outward from the elongated member at the detachable coupling.

The above as well as additional objects, features, and advantages of the present invention will become apparent in the following detailed written description.

### BRIEF DESCRIPTION OF THE DRAWINGS

The novel features believed characteristic of the invention are set forth in the appended claims. The invention itself however, as well as a preferred mode of use, further objects and advantages thereof, will best be understood by reference to the following detailed description of an illustrative embodiment when read in conjunction with the accompanying drawings, wherein:

FIGS. 1A-1B depict alternative views showing a suturing device in a loaded and a deployed disposition in accordance with one embodiment of the present invention;

FIG. 2 illustrates a suture needle assembly that may be deployed by the suturing device of the present invention;

FIGS. 3A-3F illustrate a suturing deployment sequence using the suturing device shown in FIGS. 1A-1B in accordance with a preferred embodiment of the present invention; and

FIG. 4 is a high-level flow diagram depicting steps performed during the suturing deployment sequence shown at FIGS. 3A-3F;

FIGS. 5A-5B depict alternative views showing a suturing device in a loaded and a deployed disposition in accordance with an alternative embodiment of the present invention;

FIGS. 6A-6E illustrate a suturing deployment sequence using the suturing device shown in FIGS. 5A-5B in accordance with the present invention;

FIGS. 7A-7B illustrate deployment of a needle assembly during the suturing deployment sequence shown in FIGS. 5A-5B in accordance with the present invention; and

FIG. 8 is a high-level flow diagram depicting steps performed during the suturing deployment sequence shown at FIGS. 6A-6E.

### DETAILED DESCRIPTION OF ILLUSTRATIVE EMBODIMENT(S)

The present invention is generally directed to a device for suturing relatively inaccessible surgical incision sites. The suturing device of the present invention is simple in design, inexpensive to manufacture, and easy to operate. The present invention is particularly well-suited for application to relatively inaccessible surgical sites, such as those incidental to laparoscopic and endoscopic surgery. In the embodiments depicted herein, the suturing device is advantageously implemented using a modified laparoscopic trocar. It should be noted, however, that alternate embodiments may utilize other surgical instruments which, like trocars, are routinely inserted into closed surgical procedures.

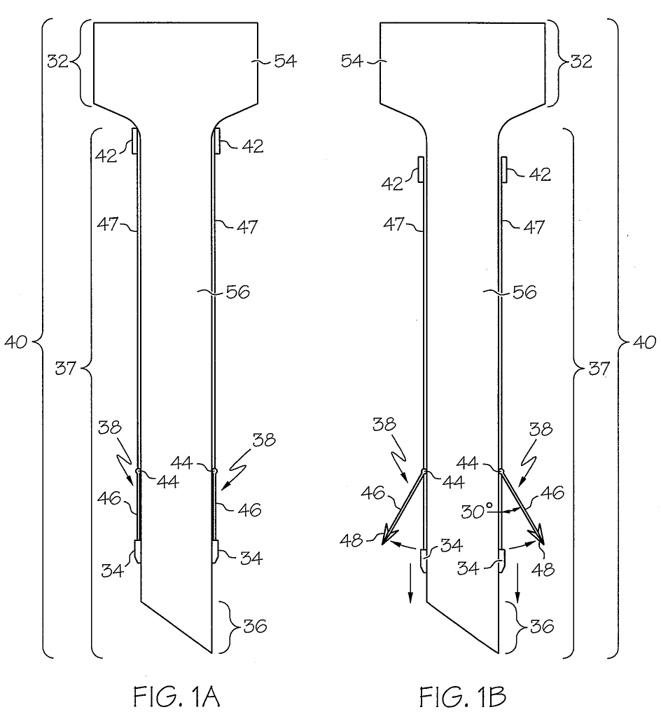

With reference now to the figures, wherein like reference numerals refer to like and corresponding parts throughout, and in particular with reference to FIGS. 1A and 1B, there are depicted alternative views showing a suturing device in the form of a trocar 40 adapted for closing puncture wounds in accordance with the present invention. Trocar 40 is particularly adapted for deploying suture needles within a peritoneal cavity or other remote surgical site. As explained in further detail below, trocar 40 includes suturing functionality for driving suture needles into tissue, such as a fascial tissue layer adjacent the puncture wound, such that sutures can be anchored on opposing edges of a surgical wound. It should be noted that while a two-needle implementation is depicted in the descriptive embodiments herein, many functional and structural aspects of the present invention may be practiced using more than two needle assemblies without departing from the spirit or scope of the present invention.

Trocar 40 generally includes an elongated tubular port member 37 having a head member 32 at its proximal end and a pointed tip 36 at its distal end. Head member 32 is contoured to serve as a manual handle and is preferably integrally coupled to port member 37, having a hollow chamber 54 communicatively coupled to the interior lumen 56 of port member 37.

Trocar 40 serves multiple roles during a minimally invasive surgical procedure such as a laparoscopy or endoscopy. In one aspect, port member 37 provides a rigid traction along the otherwise relatively “closed” surgical puncture wound with head member 32 resting outside the patient's body and pointed tip 36 disposed proximate to the surgical site. During minimally invasive surgery, surgical instruments such as laparoscopes, laparoscopic graspers, etc., are inserted through interior lumen 56 within port member 37 and into the desired surgical site for manipulation by the surgeon.

In accordance with the present invention, trocar 40 is further adapted to directly facilitate post surgical procedure suturing to close the puncture wound opened and occupied by trocar 40 during the procedure. To this end, and as depicted in FIG. 1B, trocar 40 further includes a pair of needle assemblies 38 disposed in approximate diametric opposition on the surface of port member 37. Needle assemblies 38 include suture anchors, depicted in the figures as needle heads 48, which are respectively coupled to the distal ends of needle shafts 46. Needle assemblies 38 are connected to trocar 40 at a pair of couplings 44 at the proximal ends of needle shafts 46. In a preferred embodiment, and as illustrated and explained in further detail below, couplings 44 are detachable, enabling needle assemblies 38 to be detached in response to a sufficient force applied and translated by mechanisms described herein to the back (proximal) side of needle heads 48.

FIG. 1A illustrates trocar 40 in a “loaded” position in which needle assemblies 38 are held substantially flush or in close adjacency with the exterior surface of port member 37. In the depicted embodiment, needle assemblies 38 are maintained in the retracted position by a sheath, comprising sheath members 34 that hold the distal ends of needle assemblies 38 proximate to or against the surface of port member 37. In this position, and as shown in FIG. 1A, needle shafts 46 are disposed substantially in parallel with the lengthwise axis of port member 37. As further depicted and explained below, trocar 40 is maintained in the loaded position shown in FIG. 1A during the initial surgical incision process in which the distal end of trocar 40 passes into and through the cutis and sub-cutis tissue layers.

FIG. 1B depicts trocar 40 in a “deployed” position in which the distal ends of needle assemblies 38 have been released from sheath members 34. As shown in FIG. 1B, needle assemblies 38 are elastically biased to pivot around couplings 44 and outwardly in a radial manner from port member 37. To achieve such elastic bias, couplings 44 may comprise elastic joints that integrally couple the surface material of port member 37 to the proximal ends of needle shafts 46. In other embodiments, couplings 44 comprise spring-biased hinges having material and/or structural properties for providing the depicted resilient, elastic biasing of needle assemblies 38. When released from sheath members 34, the bias applied by couplings 44 or otherwise urge needle assemblies 38 in an arc-like rotation, pivoting around couplings 44 as shown in FIG. 1B to a final extended position approximately 30° from the surface of port member 37.

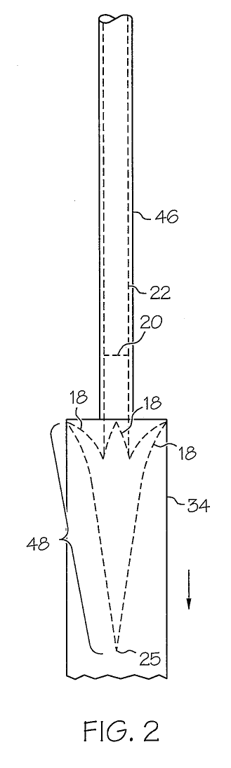

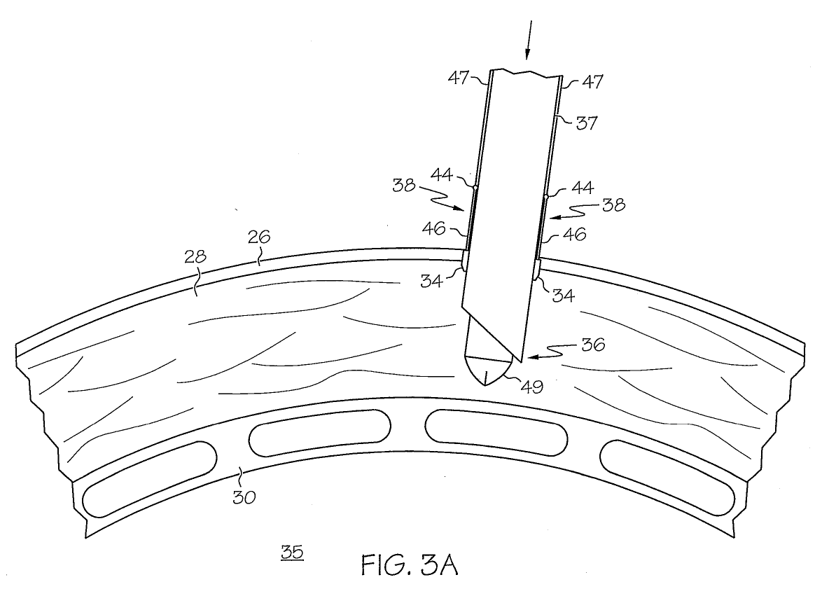

FIG. 2 illustrates a more detailed view of the constituent components of needle assemblies 38 in accordance with the present invention. FIG. 2 depicts the assembly in the loaded position with the distal end of the assembly including needle head 48 contained within sheath member 34 with a direction arrow indicating the direction of sheath withdrawal to deploy the assembly. As shown in the depicted partial phantom view, needle head 48 preferably comprises a sharply pointed primary tip 25 pointing generally toward the distal end of port member 37 and one or more barbs 18 projecting radially outward and in the opposing direction to the direction of tip 25 (i.e. toward the proximal end of port member 37). In the depicted embodiment, needle shaft 46 is hollow, having a lumen that annularly receives and retains at its distal end a proximal end 20 of needle head 48. A suture material 22 is coaxially encased and retained within the interior lumen of needle shaft 46.

With continued reference to FIGS. 1A and 1B, trocar 40 further comprises a sheath retraction assembly for driving sheath members 34 distally or proximally to switch trocar 40 between the loaded and deployed positions depicted in FIGS. 1A and 1B. The depicted sheath retraction assembly generally includes a pair of sheath retraction rods 47 having corresponding, proximally disposed latch retraction tabs 42. Retraction rods 47 are slidably disposed flush with or adjacent to the surface of port member 37, having sheath members 34 rigidly coupled at the distal ends. Retraction rods 47 transmit manual force applied to retraction tabs 42 to sheath members 34, enabling deployment of needle assemblies 38 from the loaded position shown in FIG. 1A.

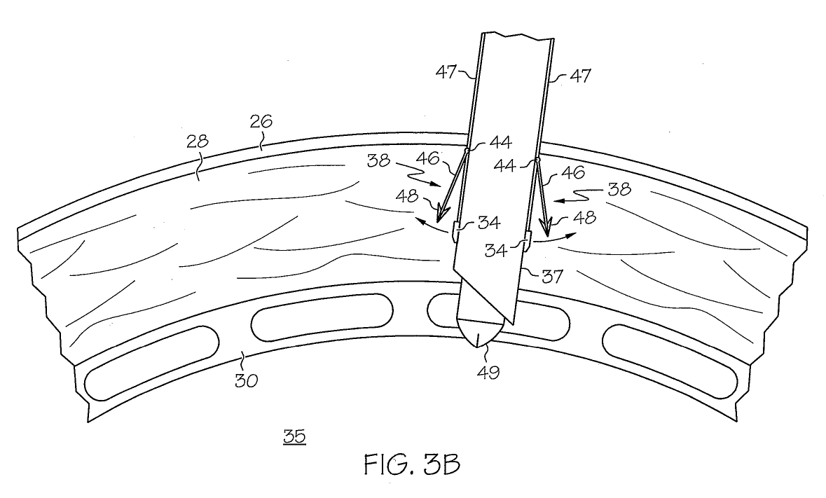

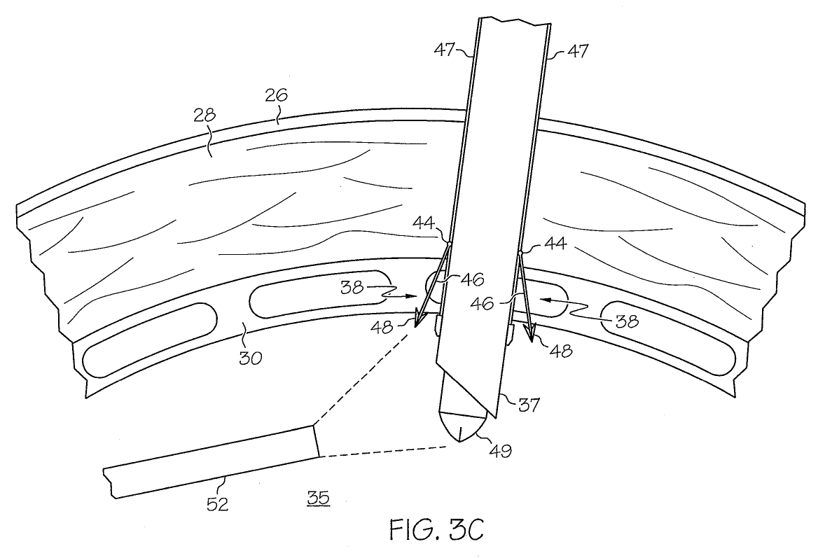

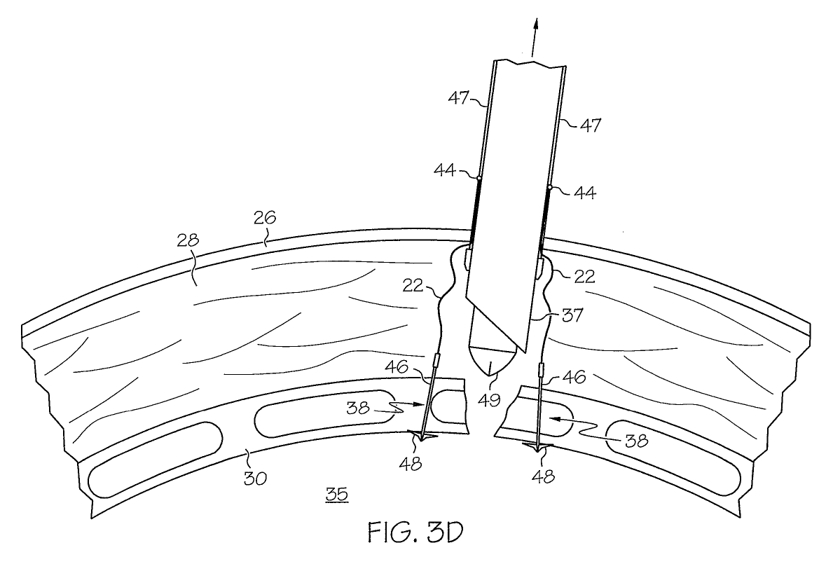

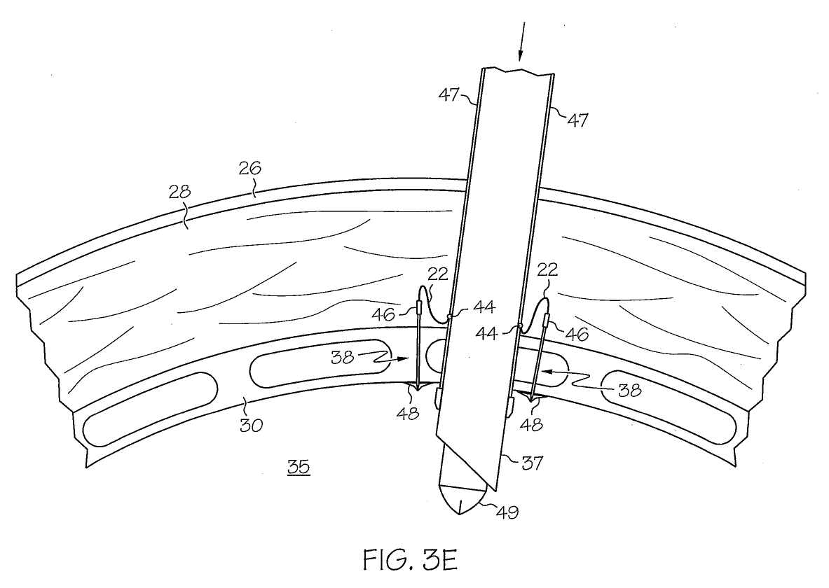

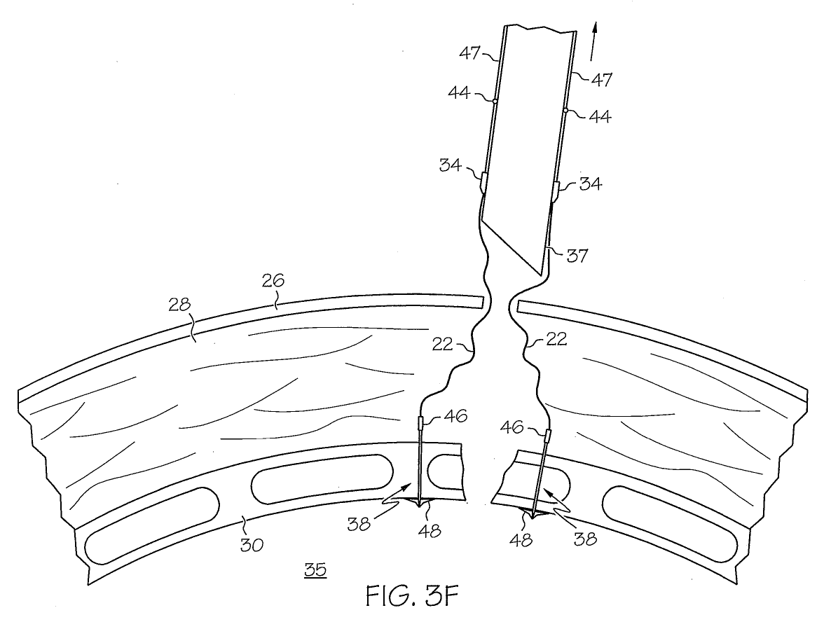

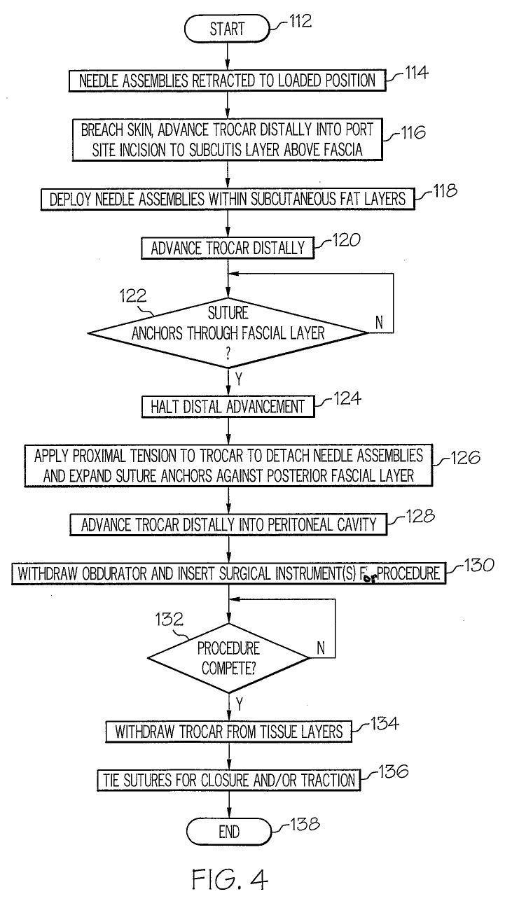

A suturing device having the features depicted in FIGS. 1 and 2 may be utilized for suturing remote surgical incision sites. As depicted and described with reference to FIGS. 3A-3F and FIG. 4, the suturing device of the present invention has features including forward deployed needle assemblies mounted near the distal end of the trocar, which render the device particularly well-suited for closing laparoscopic wounds. The deployment of the needle assemblies is coordinated with surgical procedure steps to enable the trocar utilized as the tissue penetration and/or surgical sheath to directly facilitate the subsequent wound closure or traction. It should be noted that while the embodiment shown in FIG. 1 employs a trocar as the suture deployment device, alternate embodiments may utilize other elongated surgical devices, such as obdurators, as the positioning member on which the needle assemblies may be mounted and deployed.

FIGS. 3A-3F and FIG. 4 illustrate functional aspects of the suturing device. In particular, FIGS. 3A-3F depict a sequence of deployment positions of trocar 40 within a minimally invasive surgical incision site and FIG. 4 illustrates a high-level flow diagram depicting steps performed during the trocar deployment sequence shown at FIGS. 3A-3F. The procedure begins as shown at steps 112 and 114 of FIG. 4 with needle assemblies 38 maintained retracted in the loaded position prior to the initial incision into and through the cutis or skin layer 26. Proceeding as depicted at step 116, a cutting instrument such as a scalpel is utilized to puncture or incise into and through a patient's cutis. As illustrated in FIG. 3A, following the initial incision through the cutis layer 26, the distal end of trocar 40 is advanced by the surgeon into the subcutaneous fat layer 28. The initial incision and distal advancement of trocar 40 with needle assemblies 38 in the loaded/retracted position (step 116) is carefully limited to ensure that needle assemblies 38 remain above a musculoaponeurotic layer, depicted and referred to herein as fascial layer 30, that sheaths a peritoneal cavity 35 in which the intended surgical procedure site is located.

Next, as shown at step 118 and FIG. 3B, with the distal end of trocar 40 positioned such that needle assemblies 38 are substantially immersed within subcutaneous fat layer 28 while remaining above fascial layer 30, actuator rods 47 slidably advance sheath members 34 downwardly to unsheathe the needle assembly distal ends including needle heads 48. Actuator rods 47 are advanced by manual actuation of sheath retraction tabs 42 (FIGS. 1A and 1B) to effectuate the release/deployment of needle assemblies 38 within subcutaneous fat layer 28. The deployment of needle assemblies 38 as depicted in FIG. 3B results in needle shafts 46 rotating about detachable couplings 44 through an acute angle of rotation from the surface of port member 37 such that at the end of the rotation, the tips of needle heads 48 are immersed within subcutaneous fat layer 28 and pointing angularly toward the anterior side of fascial layer 30. In a preferred embodiment, needle shafts 46 rotate 30° from the surface of port member 37 during needle assembly deployment.

An obdurator tip 49 is preferably deployed from the distal open end of port member 37 to facilitate traversal of the otherwise open distal end of trocar 40 through the various sub-cutis layers including subcutaneous fascial layer 30. Following deployment of needle assemblies 38, the surgeon resumes advancing the obdurator tipped end of trocar 40 distally through fascial layer 30 and into peritoneal cavity 35 as depicted at step 120 and FIG. 3C. As trocar 40 is advanced, needle heads 48 penetrate and breach fascial layer 30 until needle heads 48 extend into peritoneal cavity 35. As depicted in FIG. 3C, the distal advancement of trocar 40 at step 120 may be monitored by a pre-positioned laparoscope 52 within peritoneal cavity 35. Laparoscope 52 is utilized to visually monitor penetration of the distal components of trocar 40 including needle heads 48 through the posterior side fascial layer 30 and into peritoneal cavity 35. In this manner, laparoscope 52 is utilized to precisely determine the trocar advancement point at which needle heads 48 have penetrated into peritoneal cavity 35 such that trocar advancement can be halted accordingly as illustrated at steps 122 and 124.

Following a determination, such as via laparoscope 52 or otherwise, that needle heads 48 have fully penetrated fascial layer 30 and passed in peritoneal cavity 35, trocar 40 is withdrawn proximally as illustrated at step 126 and FIG. 3D. The withdrawal force applied to the body of trocar 40 applies a tension translated from anchored needle heads 48 to detachable couplings 44, resulting in detachment of needle assemblies 38 at detachable couplings 44. As farther depicted, the withdrawal force is preferably sufficient to result in expansion of the needle head barbs 18 against the inner posterior side of fascial layer 30. In this manner, detached needle assemblies are firmly anchored against the posterior side of fascial layer 30 and remain coupled to trocar 40 via sutures 22.

Following detachment and anchoring of needle assemblies 38, inward advancement of trocar 40 is resumed. As shown at step 128 and FIG. 3E, the obdurator tipped distal end of trocar 40 is advanced to a position proximate the intended surgical procedure site. With the distal end of trocar 40 in the extended position within peritoneal cavity 35, the anchored needle assemblies 38 remain loosely coupled to trocar 40 and/or external suturing mechanisms via suture material 22, which preferably comprises a bio-absorbable or otherwise biocompatible suturing material.

After the distal end of trocar 40 has been suitably advanced within peritoneal cavity 35, obdurator tip 49 is removed from trocar 40 and the minimally invasive surgical procedure, such as a laparoscopy or endoscopy, is commenced. During the procedure, port member 37 of trocar 40 is utilized as the passageway through which surgical instruments are passed to the surgical site (step 130). Upon completion of the procedure, trocar 40 is withdrawn from peritoneal cavity 35 and through tissue layers 30, 28, and 26, until trocar 40 is fully withdrawn from the puncture site as depicted at steps 132 and 134. As shown in FIG. 3F, needle assemblies 38 remain anchored by expanded needle heads 48 against the posterior fascial layer 30. In this manner, the process concludes with sutures 22 being externally manipulated and tied to provide closure or traction for the puncture through fascial layer 30 (steps 136 and 138).

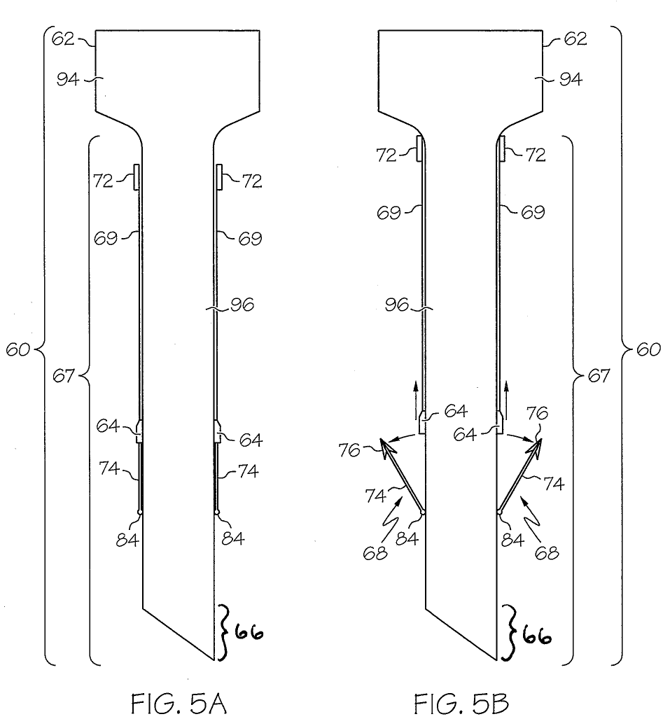

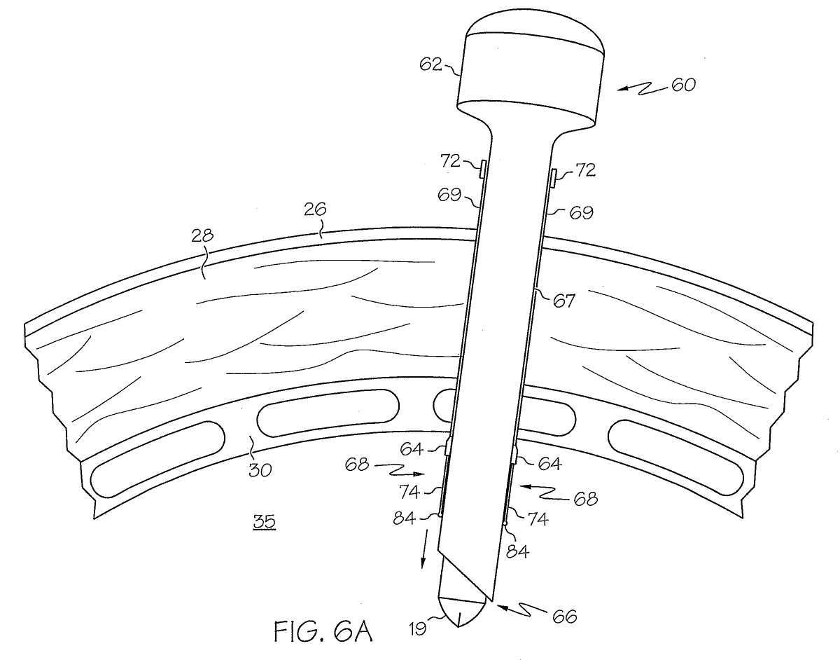

With reference to FIGS. 5A and 5B, there are depicted alternative views showing a suturing device as a modified trocar 60 adapted for closing puncture wounds in accordance with an alternate embodiment of the present invention. It should be noted that while the embodiment shown in FIGS. 5A and 5B employs a trocar as the suture deployment device, alternate embodiments may utilize other elongated surgical devices, such as obdurators, as the positioning member on which the needle assemblies may be mounted and deployed. Like trocar 40, trocar 60 is particularly adapted for deploying suture needles within a peritoneal cavity or other remote surgical site generated incident to minimally invasive surgery. Trocar 60 generally includes an elongated tubular port member 67 having a head member 62 at its proximal end and a tip 66 at its distal end. Head member 62 is contoured as a handle and is preferably integrally coupled to port member 67, having a hollow chamber 94 communicatively coupled to the interior lumen 96 of port member 67.

Trocar 60 serves multiple roles during a minimally invasive surgical procedure such as a laparoscopy or endoscopy. In one aspect, port member 67 provides a rigid traction along the otherwise relatively “closed” surgical puncture wound with head member 62 resting outside the patient's body and tip 66 disposed proximate to the surgical site. During minimally invasive surgery, surgical instruments such as laparoscopes, laparoscopic graspers, etc., are inserted through interior lumen 96 within port member 67 and into the desired surgical site for manipulation by the surgeon.

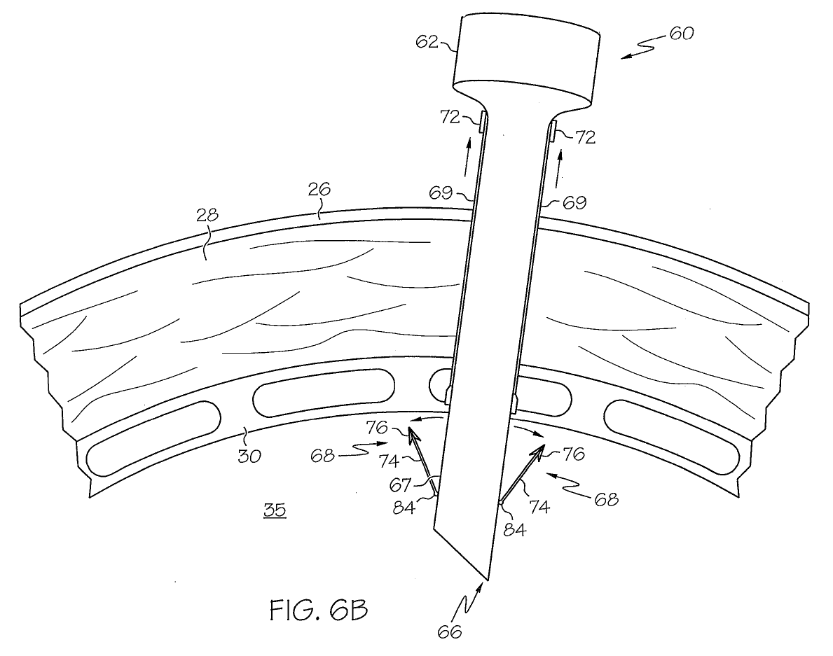

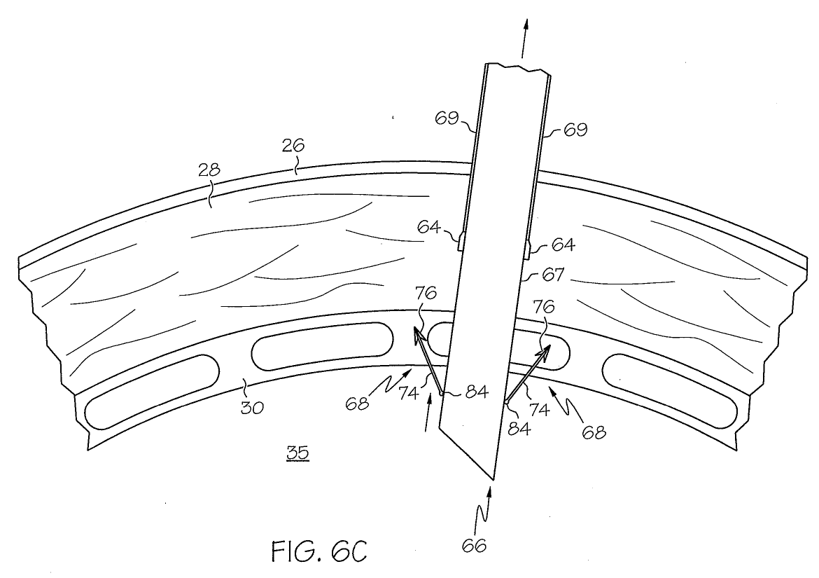

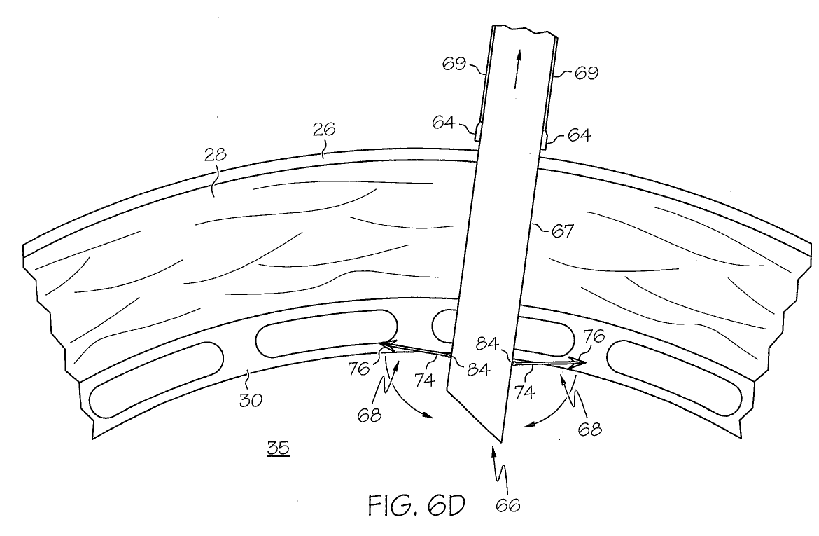

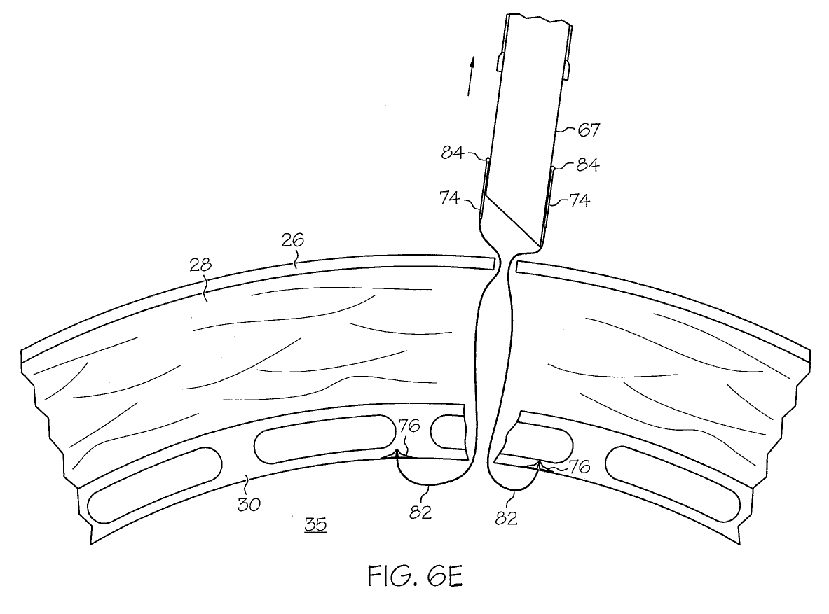

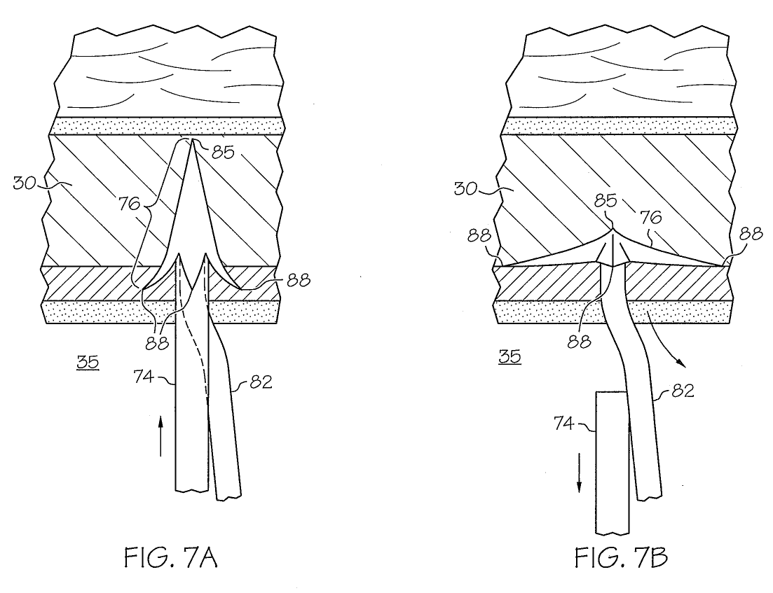

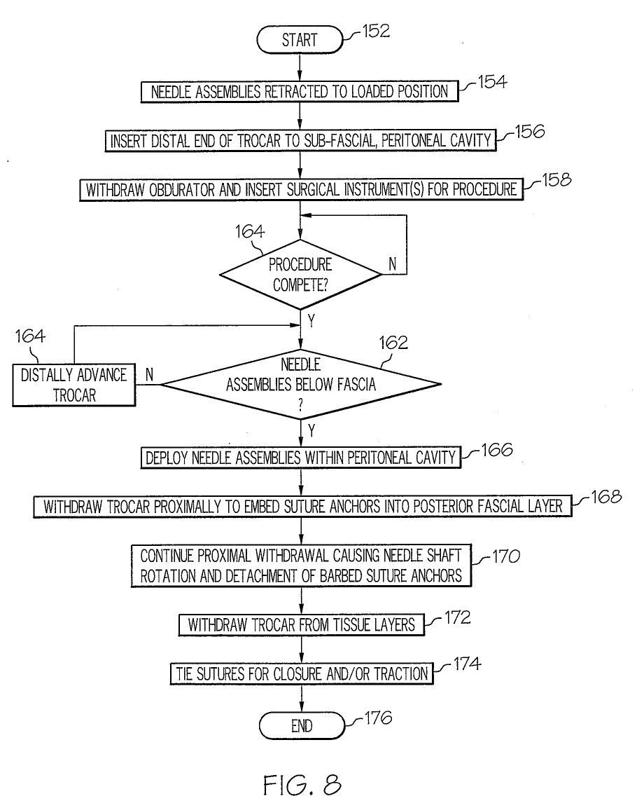

In accordance with the present invention, trocar 60 is further adapted to directly facilitate post surgical procedure suturing to close the puncture wound opened and occupied by trocar 60 during the procedure. To this end, and as depicted in FIG. 5B, trocar 60 further includes a pair of needle assemblies 68 disposed in approximate diametric opposition on the surface of port member 67. Needle assemblies 68 include suture anchors, depicted in the figures as needle heads 76, which are respectively coupled to the distal ends of needle shafts 74. Needle assemblies 68 are connected to trocar 60 at a pair of couplings 84 at the proximal ends of needle shafts 74. In one embodiment, and consistent with the suturing device and procedure depicted in FIGS. 1-4, couplings 84 may be detachable, enabling needle assemblies 68 to be detached in response to a sufficient force applied and translated by mechanisms described herein to the back (proximal) side of needle heads 76. In a preferred embodiment, and as depicted and explained in further detail with reference to FIGS. 6E, 7A, and 7B, couplings 84 are not detachable and are instead spring biased hinges enabling an angular range of motion of in excess of 180° for needle shafts 74.

FIG. 5A illustrates trocar 60 in a “loaded” position in which needle assemblies 68 are held substantially flush or in close adjacency with the exterior surface of port member 67. In the depicted embodiment, needle assemblies 68 are maintained in the retracted position by a sheath, comprising sheath members 64 that hold the distal ends of needle assemblies 68 proximate to or against the surface of port member 67. In this position, and as shown in FIG. 5A, needle shafts 74 are disposed substantially in parallel with the lengthwise axis of port member 67. As further depicted and explained below, trocar 60 is maintained in the loaded position shown in FIG. 5A during the initial surgical incision process in which the distal end of trocar 60 passes into and through the cutis and sub-cutis tissue layers.

FIG. 5B depicts trocar 60 in a “deployed” position in which the distal ends of needle assemblies 68 have been released from sheath members 64. As shown in FIG. 5B, needle assemblies 68 are disposed such that needle heads 76 point angularly in substantially the proximal direction toward head member 62. Needle assemblies 68 are elastically biased to pivot around couplings 84 and outwardly in a radial manner from port member 67. To achieve such elastic bias, couplings 84 may comprise elastic joints that integrally couple the surface material of port member 67 to the proximal ends of needle shafts 74. In other embodiments, couplings 84 comprise spring-biased hinges having material and/or structural properties for providing the depicted resilient, elastic biasing of needle assemblies 68. When released from sheath members 64, the bias applied by couplings 84 push or otherwise urge needle assemblies 68 in an arc-like rotation, pivoting around couplings 84 as shown in FIG. 5B to a final extended position approximately 30° from the surface of port member 67.

With continued reference to FIGS. 5A and 5B, trocar 60 further comprises a sheath retraction assembly for driving sheath members 64 distally or proximally to switch trocar 60 between the loaded and deployed positions depicted in FIGS. 5A and 5B. The depicted sheath retraction assembly generally includes a pair of sheath retraction rods 69 having corresponding, proximally disposed latch retraction tabs 72. Retraction rods 69 are slidably disposed flush with or adjacent to the surface of port member 67, having sheath members 64 rigidly coupled at the distal ends. Retraction rods 69 transmit manual force applied to retraction tabs 72 to sheath members 64, enabling deployment of needle assemblies 68 from the loaded position shown in FIG. 5A.

Another aspect of the present invention relates to a method for suturing remote surgical incision sites. As depicted and described with reference to FIGS. 6A-6E, FIGS. 7A-7B, and FIG. 8, the method preferably includes utilizing a modified trocar such as that depicted and described with reference to FIGS. 5A and 5B, that, in contrast to the embodiment shown in FIGS. 1A and 1B, has rearward deployed needle assemblies mounted near the distal tip of the trocar. The deployment/retraction status of the needle assemblies is coordinated with surgical procedure steps to enable the trocar utilized as the tissue penetration and/or surgical sheath instrument to directly facilitate the subsequent wound closure or traction.

FIGS. 6A-6E, FIGS. 7A-7B, and FIG. 8 illustrate additional aspects of the suturing device and method in accordance with the alternate embodiment of the present invention. FIGS. 6A-6E depict a deployment sequence of trocar 60 within a minimally invasive surgical incision site, and FIG. 8 illustrates a high-level flow diagram depicting steps performed during the deployment sequence. The process begins as shown at steps 152 and 154 of FIG. 8 with needle assemblies 68 maintained retracted in the loaded position prior to the initial advancement of the device into and through the cutis 26. Proceeding as depicted at step 156, a cutting instrument such as a scalpel is utilized to make an incision through a patient's cutis 26. As illustrated in FIG. 6A, following the initial advancement through the cutis layer 26, the distal end of trocar 60 is advanced by the surgeon into and through the subcutaneous fat layer 28 and fascial layer 30. An obdurator tip 19 is preferably deployed from the distal open end of port member 67 to facilitate traversal of the otherwise open distal end of trocar 60 through fascial layer 30. The advancement of trocar 60 with needle assemblies 68 in the loaded position continues until, as shown in FIG. 6A, the distal end has penetrated into the sub-fascial, peritoneal cavity 35 in preparation for the surgical procedure. The obdurator tip 19 is subsequently withdrawn and port member 67 provides a rigid sheath through which surgical instruments (not depicted) may be passed during the procedure (step 158).

Following the procedure, and with the distal end of trocar 60 including needle assemblies 68 advanced distally below fascial layer 30, retraction tabs 72 are actuated to urge actuator rods 69 proximally to unsheathe needle heads 76, thus deploying needle assemblies 68 within peritoneal cavity 35 as shown at steps 160, 162, 164, and 166 and FIG. 6B. Actuator rods 69 are withdrawn by manual actuation of retraction tabs 72 to effectuate the release/deployment of needle assemblies 68 within peritoneal cavity 35. The deployment of needle assemblies 68 as depicted in FIG. 6B results in needle shafts 74 rotating about couplings 84 through an acute angle of rotation from the surface of port member 67 such that at the end of the rotation, the tips of needle heads 76 are immersed within peritoneal cavity 35 and pointing angularly toward the posterior side of fascial layer 30. In a preferred embodiment, needle shafts 74 rotate 30° from the surface of port member 67 during needle assembly deployment.

Next, as depicted at step 168, trocar 60 is withdrawn proximally to embed the suture anchor needle heads 76 into the posterior side of fascial layer 30. FIGS. 6C and 7A provide a more detailed illustration of the initial fascial penetration of needle heads 76. Specifically, FIG. 6C illustrates the deployed orientation of needle assemblies 68 in which needle shafts 74 remain positioned in an acute angle (approximately 30°) with respect to the surface of port member 67 with needle heads 76 pointed rearwardly, or proximally, with respect to port member 67 during the initial penetration of needle heads 76. As shown in FIG. 7A in conjunction with 6C, the primary tip 85 of each of needle heads 76 has penetrated substantially though the posterior fascial layer 30 until a set of radially projecting barbs 88 has also penetrated the posterior side. Each of needle shafts 74 includes a hollow distal end in contact with the proximal end of respectively needle heads 76. As further shown in FIG. 7A, the hollow distal end of needle shaft 74 is adapted for coaxially encasing a portion of suture material 82 which is terminally coupled to needle head 76.

Proceeding as shown at step 170 and FIGS. 6D, 6E, and 7B, the proximal withdrawal of trocar 60 through the tissue layers results in a continuing rotation of needle shafts 74 and ultimate detachment of the barbed needle heads 76 from needle shafts 74. Specifically, and as illustrated in FIG. 6D, the post-anchoring rotation of needle shafts 74 continues at couplings 84 and another rotation begins at the points at which needle heads 76 are initially embedded within fascial layer 30.

The withdrawal force applied to the body of trocar 60 is translated to the rotational tension translated to the anchored needle heads 76, resulting in detachment of needle heads 76 at a frangible or otherwise detachable joint or coupling between needle heads 76 and corresponding needle shafts 74 as shown at FIG. 6E. As further depicted in FIGS. 7B and 6E, the withdrawal force is preferably sufficient to result in expansion of the needle head barbs 88 within the fascial layer 30. In this manner, needle heads 76 are firmly anchored against the posterior side of fascial layer 30 and remain coupled to trocar 60 via sutures 82.

Following detachment and anchoring of needle heads 76, proximal withdrawal of trocar 60 continues from peritoneal cavity 35 and through tissue layers 30, 28, and 26, until trocar 60 is fully withdrawn from the puncture site as depicted at step 172. As shown in FIGS. 6E and 7B, needle heads 76 remain anchored within the fascial layer 30. The suturing procedure concludes with sutures 82 being externally manipulated and tied to provide closure or traction for the puncture through fascial layer 30 (steps 174 and 176).

While the invention has been particularly shown and described with reference to a preferred embodiment, it will be understood by those skilled in the art that various changes in form and detail may be made therein without departing from the spirit and scope of the invention. These alternate implementations all fall within the scope of the invention.

What is claimed is:

1. A surgical incision suturing device comprising:
an elongated member having a distal end adapted for placement within a surgical site; and
a pair of needle assemblies disposed in substantially diametric opposition on the outside surface of said elongated member, wherein each of said needle assemblies comprises a suture anchor coupled to the distal end of a needle shaft, and wherein each of said needle assemblies is detachably coupled to said elongated member at a detachable coupling that couples the proximal end of the needle shaft to said elongated member, said needle assemblies biased to pivot radially outward from said elongated member at the detachable coupling.

2. The suturing device of claim 1, said needle shaft having a hollow distal end in communication with the proximal end of said suture anchor, said hollow distal end adapted for coaxially encasing a portion of suture material.

3. The suturing device of claim 1, wherein each of said suture anchors comprises a barbed needle head having a sharply pointed tip.

4. The suturing device of claim 3, wherein said pointed tip points toward the distal end of said elongated member when said sheath houses the distal ends of said needle assemblies.

5. The suturing device of claim 3, wherein said pointed tip points toward the proximal end of said elongated member when said sheath houses the distal ends of said needle assemblies.

6. The suturing device of claim 3, wherein each of said barbed needle heads comprises at least one barb located proximal to said tip and projecting radially outward from said barbed needle head.

7. The suturing device of claim 6, said wherein said barb comprises a flexible material that expands further radially outward responsive to tension applied from the attached suture and transferred to said barb.

8. 9. The suturing device of claim 1, wherein the detachable coupling comprises a frangible joint.

10. The suturing device of claim 1, wherein said elongated member is a substantially rigid tubular cannula.

11. The suturing device of claim 10, wherein said tubular port member is a trocar.

12. The suturing device of claim 1, further comprising:
a sheath operable in a loaded position to releasably restrain said needle assemblies such that said needle shafts are disposed substantially in parallel with the lengthwise axis of said elongated member; and
an actuator operable to actuate said sheath from the loaded position to a deployed position in which said needle assemblies are released from said sheath.

13. The suturing device of claim 12, wherein said sheath is adapted to house at least a portion of the distal ends of said needle assemblies

14. The suturing device of claim 13, wherein said actuator is operable to actuate said sheath between said loaded position and said deployed position.

15. The suturing device of claim 13, wherein said sheath comprises a pair of sheath members that individually house at least a portion of the distal end of each of said needle assemblies.

16. A surgical incision suturing device comprising:
an elongated member having a distal end adapted for placement within a surgical site; and
a pair of needle assemblies disposed in substantially diametric opposition on the outside surface of said elongated member, wherein:
each of said needle assemblies comprises a suture anchor coupled to the distal end of a needle shaft;
said needle assemblies are biased to pivot radially outward from said elongated member at the detachable coupling; and
each of said suture anchors comprises a barbed needle head having a sharply pointed tip that points toward the distal end of said elongated member when said sheath houses the distal ends of said needle assemblies.
